(19) United States
(12) Patent Application Publication
(10) Pub. No.: US 2025/0268543 A1
(43) Pub. Date: Aug. 28, 2025

(54) X-RAY COMPUTED TOMOGRAPHY APPARATUS, INFORMATION PROCESSING METHOD, INFORMATION PROCESSING APPARATUS, AND STORAGE MEDIUM

(71) Applicant: CANON MEDICAL SYSTEMS CORPORATION, Otawara-shi (JP)

(72) Inventors: Kosuke ARITA, Nasushiobara (JP); Asateru KIMURA, Otawara (JP)

(73) Assignee: CANON MEDICAL SYSTEMS CORPORATION, Otawara-shi (JP)

(21) Appl. No.: 19/060,875

(22) Filed: Feb. 24, 2025

(30) Foreign Application Priority Data
Feb. 28, 2024 (JP) 2024-028694

(51) Int. Cl. A61B 6/03; A61B 5/00; A61B 5/055; A61B 6/42; A61B 8/00

(52) U.S. Cl. CPC A61B 6/032; A61B 5/0033; A61B 5/055; A61B 6/4241; A61B 8/5215

(57) ABSTRACT

An X-ray CT apparatus of an embodiment includes processing circuitry. The processing circuitry configured to acquire a number of photons by performing a CT scan on a target event, generate a first feature amount from the number of photons, match the first feature amount to second feature amounts generated using a machine learning model from the number of photons of a reference event, the number of photons of the reference event being a number of photons generated from known component amounts of the reference event and having a nonlinear relationship with the known component amounts, and estimate the known component amounts corresponding to the second feature amount matched to the first feature amount as component amounts of the target event.

### CROSS-REFERENCE TO RELATED APPLICATION

Priority is claimed on Japanese Patent Application No. 2024-028694, filed Feb. 28, 2024, the content of which is incorporated herein by reference.

### FIELD

Embodiments disclosed in the present specification and drawings relate to an X-ray computed tomography (CT) apparatus, an information processing method, an information processing apparatus, and a storage medium.

### BACKGROUND

In diagnosis, it is important to estimate component amounts inside a subject from an image of the subject. In this regard, a technology for measuring component amounts inside skin from an image of the skin is known.

However, when a discrepancy occurs between an actual phenomenon and a simulation result, a component in a living body which is far from a correct answer may be estimated. In particular, this discrepancy may be a black box that cannot be expressed by an equation or an expression by an equation cannot be specified, and it may be difficult to correct the discrepancy. As a result, a state different from an actual state of a subject may be estimated, and a correct diagnosis may not be made.

In addition, scenes in which a black box discrepancy occurs between the actual phenomenon and the simulation result may occur not only in a medical field, but also in any field such as physics, chemistry, engineering, biology, Earth science, information, finance, and economics. Therefore, the above problem is common to all fields in which a discrepancy may occur between an actual phenomenon and a simulation result.

### BRIEF DESCRIPTION OF THE DRAWINGS

FIG. 1 is a diagram which represents a configuration example of an information processing apparatus in an embodiment.

FIG. 2 is a flowchart which represents a flow of a series of processing in a processing circuit according to an embodiment.

FIG. 3 is a diagram for describing a method of acquiring a real spectral reflectance.

FIG. 4 is a diagram for describing a method of generating a real feature vector.

FIG. 5 is a diagram for describing a method of generating a pseudo spectral reflectance.

FIG. 6 is a diagram for describing a method of generating a pseudo feature vector.

FIG. 7 is a diagram for describing feature vector matching.

FIG. 8 is a diagram which represents an example of a matching result.

FIG. 9 is a diagram which represents an example of a display screen showing a map of internal component amounts in a living organism.

FIG. 10 is a diagram showing an example of an X-ray CT apparatus 1 according to an embodiment.

FIG. 11 is a diagram showing an example of a configuration of the DAS 16 according to the embodiment.

### DETAILED DESCRIPTION

An X-ray CT apparatus, an information processing method, an information processing apparatus, and a storage medium according to an embodiment will be described below with reference to the drawings.

The information processing method according to an embodiment includes acquiring first observation data, which is observation data of a target event, generating a first feature amount based on the first observation data using deep distance learning, matching the first feature amount with a second feature amount generated using deep metric learning based on second observation data that is the observation data of a reference event generated based on a second component amount, which is a known component amount of the reference event, and which has a nonlinear relationship with the second component amount, and estimating the second component amount corresponding to the second feature amount matched with the first feature amount as a first component amount, which is a component amount of the target event. Through such processing, it is possible to estimate information with high accuracy even when there is a discrepancy between an actual phenomenon and a simulation result.

The X-ray CT apparatus include processing circuitry. The processing circuitry is configured to acquire a number of photons by performing a CT scan on a target event. The processing circuitry is configured to generate a first feature amount from the number of photons. The processing circuitry is configured to match the first feature amount to second feature amounts generated using a machine learning model from the number of photons of a reference event, the number of photons of the reference event being a number of photons generated from known component amounts of the reference event and having a nonlinear relationship with the known component amounts. The processing circuitry is configured to estimating the known component amounts corresponding to the second feature amount matched to the first feature amount as component amounts of the target event. Through such processing, it is possible to estimate information with high accuracy even when there is a discrepancy between an actual phenomenon and a simulation result.

### Overview

The information processing apparatus of the embodiment acquires observation data obtained when a target event is observed. For example, the target event is “edema” in a medical field, and the observation data is “spectral reflectance” obtained from an image of skin in which edema has occurred. Note that the target event is not limited to edema, and may be other symptoms or diseases. Furthermore, the target event is not limited to a medical field, and may be an object to be analyzed in other fields, such as physics, chemistry, engineering, biology, earth science, information, finance, and economics. In the present embodiment, the target event is described as “edema” as an example.

When the information processing apparatus of the embodiment acquires an image of skin in which edema has occurred (hereinafter also referred to as an edema image), it uses deep metric learning to generate a feature vector based on a spectral reflectance (hereinafter referred to as a real spectral reflectance) corresponding to each pixel value of the edema image. In the following description, a feature vector derived from the real spectral reflectance is referred to as a real feature vector. The real spectral reflectance obtained from the edema image is an example of the “first observation data,” and the real feature vector is an example of the “first feature amount.”

The information processing apparatus of the embodiment uses a simulation model (a decoder MDL2 to be described below) to generate a pseudo-spectral reflectance obtained when a reference event (for example, edema) is observed based on a known internal component amount in a living organism of the reference event. Hereinafter, the known internal component amount in a living organism is referred to as a pseudo internal component amount, and the pseudo-generated spectral reflectance is referred to as a pseudo spectral reflectance. This pseudo spectral reflectance is labeled with the pseudo internal component amount in a living organism. The pseudo internal component amount is an example of the “second component amount,” and the pseudo spectral reflectance is an example of the “second observation data.”

The internal component amount in a living organism may include various component amounts such as C_{Hb}, C_{H2O}, and Cₘₑₗ. C_{Hb} is a hemoglobin concentration [g/L] contained in the dermis, C_{H2O} is a water concentration [g/L] contained in subcutaneous tissue, and Cₘₑₗ is a melanin concentration [g/L] contained in the epidermis. In other words, the internal component amount in a living organism is a component amount set that is a combination of a plurality of component amounts. Units of each of the internal component amount in a living organism are not limited to [g/L], and various units indicating proportions such as [%] may be adopted.

In the embodiment, the spectral reflectance and the internal component amount in a living organism have a nonlinear relationship with each other. In a typical example, the spectral reflectance changes more rapidly as the hemoglobin concentration C_{Hb} is reduced, and the spectral reflectance changes more gradually as the hemoglobin concentration C_{Hb} is increased.

The information processing apparatus of the embodiment uses deep metric learning to generate a feature vector based on the pseudo spectral reflectance labeled with the pseudo internal component amount in a living organism. Hereinafter, a feature vector derived from the pseudo spectral reflectance is referred to as a pseudo feature vector. The pseudo feature vector is an example of a “second feature amount.”

The information processing apparatus of the embodiment moves the real feature vector from a first feature space in which the real feature vector is distributed to a second feature space in which the pseudo feature vector is distributed.

The information processing apparatus of the embodiment matches the real feature vector with the pseudo feature vector in the second feature space.

The information processing apparatus of the embodiment then estimates a pseudo internal component amount in a living organism corresponding to the pseudo feature vector matched with the real feature vector as the internal component amount in a living organism of the target event (hereinafter referred to as a real internal component amount in a living organism). The real internal component amount in a living organism is an example of the “first component amount.”

In this manner, by embedding a real spectral reflectance and a pseudo spectral reflectance as feature vectors in the feature space using deep metric learning, and further matching the real feature vector with the pseudo feature vector in the feature space, it is possible to estimate information with high accuracy even when a relationship between an actual phenomenon and a simulation result cannot be expressed mathematically. For example, it is possible to estimate an extent of the internal component amount in a living organism, which indicates a degree of edema in the living organism, while reducing an influence of a discrepancy between the actual phenomenon and the simulation result.

In other words, by embedding a real spectral reflectance and a pseudo spectral reflectance as feature vectors in the feature space using deep metric learning, and further matching the real feature vector and the pseudo feature vector in the feature space, it is possible to estimate the real internal component amount in a living organism of a patient based on an image of edema of the patient, and furthermore, it is possible to more accurately diagnose the patient based on the real internal component amount in the living organism.

### Configuration of Information Processing Apparatus

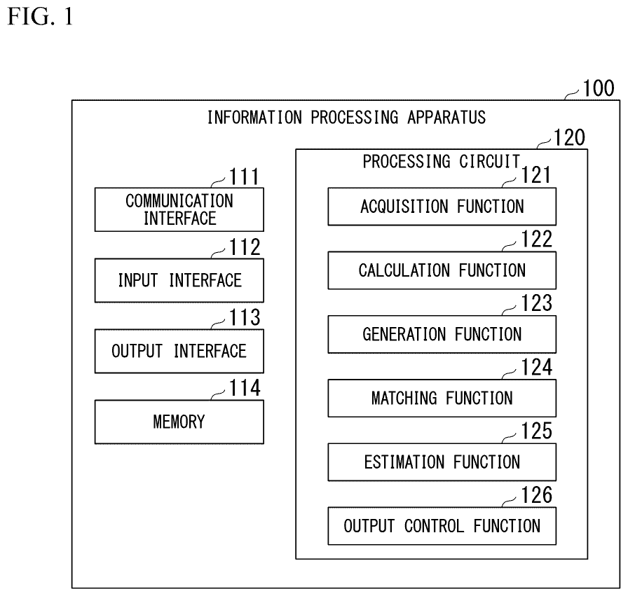

FIG. 1 shows an example of a configuration of an information processing apparatus 100 in an embodiment. The information processing apparatus 100 includes, for example, a communication interface 111, an input interface 112, an output interface 113, a memory 114, and a processing circuit 120.

The communication interface 111 communicates with an external device via a communication network NW. The communication network NW may refer to a general information and communication network that uses electrical communication technology. For example, the communication network NW includes wireless or wired local area networks (LANs) such as a hospital backbone LAN and the Internet, as well as telephone communication line networks, optical fiber communication networks, cable communication networks, and satellite communication networks. The communication interface 111 includes, for example, a network interface card (NIC) and an antenna for wireless communication.

The input interface 112 receives various input operations from an operator, converts the received input operations into electrical signals, and outputs the electrical signals to the processing circuit 120. For example, the input interface 112 includes a mouse, a keyboard, a trackball, a switch, a button, a joystick, a touch panel, and the like. The input interface 112 may be, for example, a user interface that receives a voice input from a microphone or the like. When the input interface 112 is a touch panel, the input interface 112 may also have a display function of a display 113a included in the output interface 113 which will be described below.

Note that in this specification, the input interface 112 is not limited to an interface that includes physical operating parts such as a mouse and a keyboard. For example, an example of the input interface 112 includes an electrical signal processing circuit that receives an electrical signal corresponding to an input operation from an external input device provided separately from the device and outputs the electrical signal to a control circuit.

The output interface 113 includes, for example, a display 113a and a speaker 113b. The display 113a displays various types of information. For example, the display 113a displays images generated by the processing circuit 120 and a graphical user interface (GUI) for receiving various input operations from an operator. For example, the display 113a is a liquid crystal display (LCD), a cathode ray tube (CRT) display, an organic electroluminescence (EL) display, or the like. The speaker 113b outputs information input from the processing circuit 120 as sound.

The memory 114 is realized by, for example, a semiconductor memory element such as a random access memory (RAM), a flash memory, a hard disk, or an optical disk. These non-transient storage media may be realized by other storage devices connected via a communication network NW, such as a network attached storage (NAS) or an external storage server device. The memory 114 may also include a non-transient storage medium such as a read only memory (ROM) or a register. The memory 114 stores programs executed by the hardware processor of the processing circuit 120, various calculation results by the processing circuit 120, model information, and the like.

The model information is information (a program or an algorithm) that defines an autoencoder including an encoder MDL1 and a decoder MDL2, which will be described below, and a first deep metric learning model MDL3-1 and a second deep metric learning model MDL3-2 for generating a feature vector. MDL is simply a code that represents an abbreviation of MODEL.

The processing circuit 120 includes, for example, an acquisition function 121, a calculation function 122, a generation function 123, a matching function 124, an estimation function 125, and an output control function 126. The processing circuit 120 realizes these functions by, for example, a hardware processor (computer) executing a program stored in the memory 114 (storage circuit). The acquisition function 121 is an example of an “acquisition unit,” the calculation function 122 is an example of a “calculation unit,” the generation function 123 is an example of a “generation unit,” the matching function 124 is an example of a “matching unit,” and the estimation function 125 is an example of an “estimation unit.”

A hardware processor in the processing circuit 120 refers to a circuit such as a central processing unit (CPU), a graphics processing unit (GPU), an application specific integrated circuit (ASIC), a programmable logic device (for example, a simple programmable logic device (SPLD) or a complex programmable logic device (CPLD), or a field programmable gate array (FPGA). Instead of storing a program in the memory 114, the program may be directly embedded into a circuit of the hardware processor. In this case, the hardware processor realizes its function by reading and executing the program embedded into the circuit. The program described above may be stored in the memory 114 in advance, or may be stored in a non-transitory storage medium such as a DVD or CD-ROM and installed in the memory 114 from the non-transient storage medium by the non-transitory storage medium being attached to a drive device (not shown) of the information processing apparatus 100. The hardware processor is not limited to being configured as a single circuit, but may be configured as a single hardware processor by combining a plurality of independent circuits to realize each function. In addition, a plurality of components may be integrated into a single hardware processor to realize each function.

### Processing Flow of Information Processing Apparatus

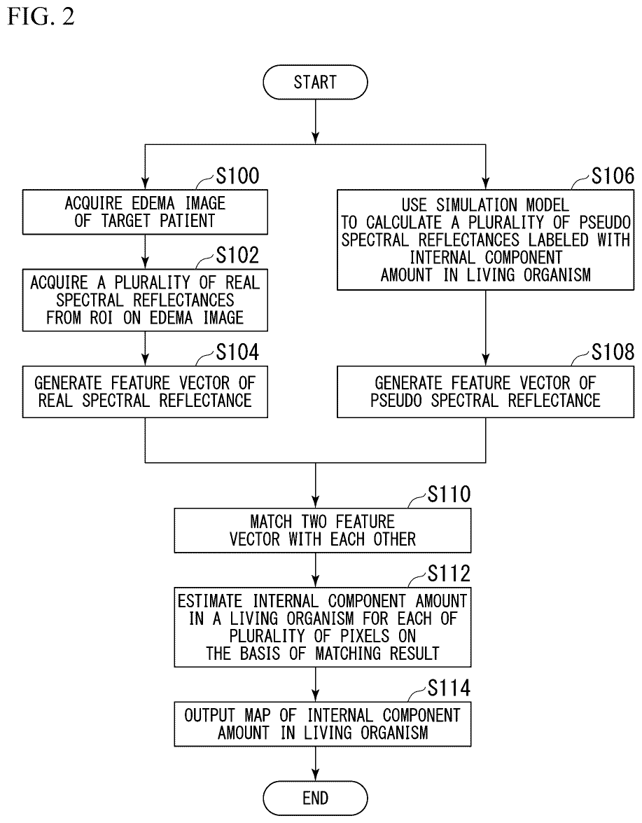

A series of processing by the processing circuit 120 of the information processing apparatus 100 will be described below with reference to a flowchart. FIG. 2 is a flowchart which shows a flow of the series of processing by the processing circuit 120 according to the embodiment. Processing of S100 to S108 in this flowchart correspond to pre-processing for performing feature vector matching, which will be described below, and processing of S110 to S114 correspond to main processing including feature vector matching.

First, the acquisition function 121 acquires an edema image IMG of a target patient (step S100).

The edema image IMG is, for example, an image of skin captured by a camera that visualizes a real spectral reflectance R of the skin at a site where edema may be present. The edema image IMG may be, for example, a three-dimensional image represented by a width x, a height y, and a wavelength λ at which the image is visualized. A pixel value of each pixel in the edema image IMG is the real spectral reflectance R.

The camera used to generate the edema image IMG is typically a multispectral camera that visualizes the real spectral reflectance R of a plurality of wavelength bands (spectra), but is not limited to this and may be a camera that visualizes only the real spectral reflectance R of a single wavelength band.

For example, the acquisition function 121 may access a database, which is an external device, via the communication interface 111 and acquire the edema image IMG from the database. In addition, when a doctor of a patient or the like inputs the edema image IMG into the input interface 112, the acquisition function 121 may acquire the edema image IMG from the input interface 112. Moreover, when the edema image IMG is stored in the memory 114, the acquisition function 121 may acquire the edema image IMG from the memory 114. Furthermore, when the patient takes an image of his or her own edema with a camera at home or the like, the acquisition function 121 may access the camera via the communication interface 111 and acquire the edema image IMG from the camera.

In addition to or instead of acquiring an edema image IMG captured by a camera, the acquisition function 121 may acquire absorbance of a skin surface detected by a wearable sensor attached to the patient's arm, leg, or the like as the edema image IMG.

When the acquisition function 121 acquires the edema image IMG, it may perform image processing such as smoothing filtering or edge extraction on the edema image IMG. This makes it possible to remove palm prints and body hair that are reflected in the edema image IMG, and to more accurately extract edema features from the edema image IMG.

Next, the acquisition function 121 acquires the real spectral reflectance R from each of the many pixels on the edema image IMG (step S102).

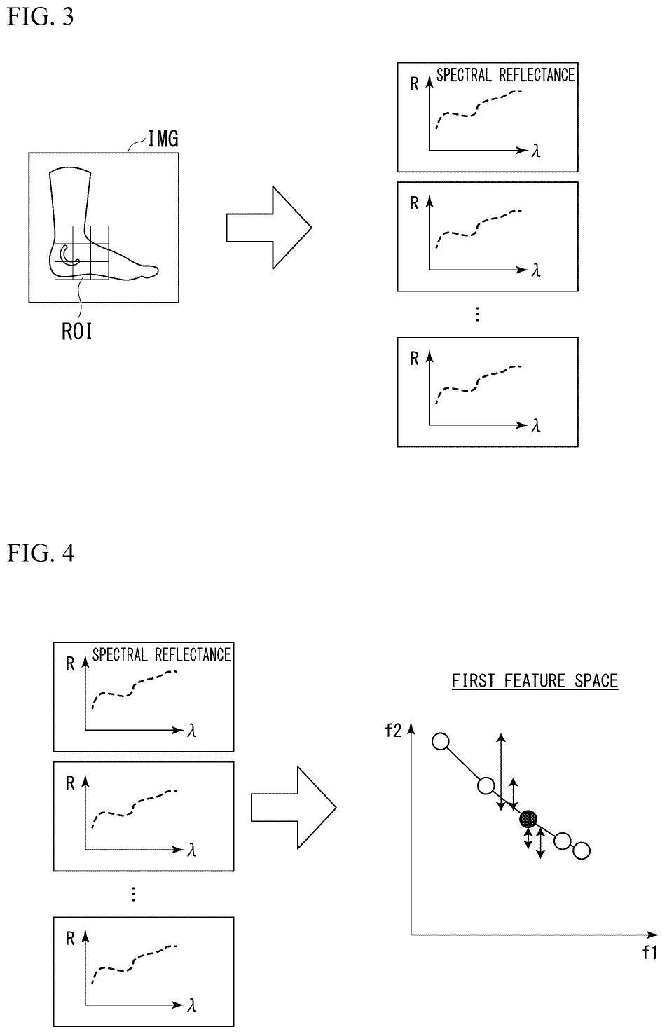

FIG. 3 is a diagram for describing a method for acquiring the real spectral reflectance R. As shown in the figure, an ROI (Region of Interest) may be set on the edema image IMG. The ROI may be set manually by a user or automatically based on edema features (edges, brightness, etc.). When the ROI is set on the edema image IMG, the acquisition function 121 acquires the real spectral reflectance R from each of the many pixels included in the ROI. For example, when the ROI includes nine pixels, nine real spectral reflectances R are acquired.

Returning to the description of the flowchart, the generation function 123 next converts each of the plurality of real spectral reflectances R obtained from the ROI on the edema image IMG into a real feature vector using deep metric learning (step S104). In other words, the generation function 123 generates a real feature vector based on each of the plurality of real spectral reflectances R using deep metric learning.

FIG. 4 is a diagram for describing a method for generating a real feature vector. For example, the generation function 123 may use the first deep metric learning model DL3-1 that has been learned in advance to embed each of the plurality of real spectral reflectances R into the first feature space as a real feature vector. In the shown example, the first feature space is represented as a two-dimensional space of f1 and f2, but is not limited to this. For example, the first feature space may be a high-dimensional space of three or more dimensions.

The first deep metric learning model MDL3-1 is a machine learning model that is learned to generate a real feature vector based on the real spectral reflectance R (to embed the real spectral reflectance R into the first feature space) when the real spectral reflectance R is input. The first deep metric learning model MDL3-1 is learned to dispose similar real feature vectors close to each other and dissimilar real feature vectors far from each other in the first feature space when the real spectral reflectance R is embedded in the first feature space. For example, the first deep metric learning model MDL3-1 is implemented by a neural network.

Generally, in deep metric learning, labels of positive pairs (for example, two identical component amounts in a living organism) and negative pairs (for example, two different component amounts in a living organism) are prepared for a measured value (for example, a spectral reflectance), and feature vectors labeled with positive pairs are learned to be closer to each other in the feature space and feature vectors labeled with negative pairs are learned to be far from each other.

In the present embodiment, the real internal component amount in a living organism corresponding to the real spectral reflectance R are largely unknown and cannot be used as a label. Therefore, by adding another set of dataset of the real spectral reflectance R and the real internal component amount in a living organism through data expansion, the first deep metric learning model MDL3-1 may be learned by self-supervised learning with the two datasets as a positive pair.

The real spectral reflectance R added through data expansion may be subjected to signal processing that reproduces noise generated during capturing an image. For example, the real spectral reflectance R may be uniformly reduced at all wavelengths to reproduce reflection of shadows. In addition, the real spectral reflectance R may be changed only at a specific wavelength to reproduce noise of the camera sensor.

The generation function 123 inputs the real spectral reflectance R to the first deep metric learning model MDL3-1 learned in this manner. As a result, the first deep metric learning model MDL3-1 generates a real feature vector in response to the input of the real spectral reflectance R, and further disposes similar real feature vectors close to each other in the first feature space.

In general, the real spectral reflectance R is likely to contain noise due to various factors such as reflected light from a skin surface, the reflection of shadows, and variation in melanin concentration. When such noise is contained, the real spectral reflectance R may be large or small even if the hemoglobin concentration C_{Hb} in the skin is the same. In other words, even if the hemoglobin concentration C_{Hb} is the same, sensitivity of the real spectral reflectance R will not match.

In contrast, in the present embodiment, the real spectral reflectance R is converted into a real feature vector by deep metric learning (the real spectral reflectance R is embedded in the first feature space), so that an effect of noise can be reduced. As a result, by matching feature vectors which will be described below, it is possible to estimate with high accuracy the internal component amount in a living organism, such as the hemoglobin concentration C_{Hb}, from the edema image IMG.

When an image of edema is captured after removing factors such as reflected light from a skin surface, the reflection of shadows, and variations in melanin concentration, the edema image IMG will not contain noise. In this case where noise is not contained, the real spectral reflectance R of the edema image IMG may be used as it is for matching described below without being converted into a real feature vector.

Returning to the description of the flowchart, the calculation function 122 uses the simulation model to calculate a plurality of pseudo spectral reflectances R\hat{ } labeled with known internal component amount in a living organism (that is, a pseudo internal component amount in a living organism) (step S106).

The simulation model may be, for example, the latter decoder MDL2 among the encoder MDL1 and the decoder MDL2 included in the autoencoder.

The encoder MDL1 is a machine learning model in a front part of the autoencoder, and converts the real spectral reflectance R into a pseudo internal component amount in a living organism. The pseudo internal component amount in a living organism output by the encoder MDL1 corresponds to a so-called latent variable.

The encoder MDL1 may be implemented by, for example, a neural network learned to make an output result of the decoder MDL2 (the pseudo spectral reflectance R\hat{ } of each pixel of an edema image of a patient) approach the real spectral reflectance R of each pixel of the edema image of the patient. The encoder MDL1 may be implemented using a genetic algorithm instead of the neural network. Furthermore, the encoder MDL1 may be implemented using other optimization methods such as Bayesian optimization, grid search, random search, CMA-ES, a Nelder-Mead method, and a quasi-Newton method.

The decoder MDL2 is a numerical calculation model that calculates or simulates the pseudo spectral reflectance R\hat{ } of each pixel of the edema image of a patient based on the internal component amount in a living organism (C_{Hb}, C_{H2O}, Cₘₑₗ, . . . ) of the patient. The decoder MDL2 may be implemented on the basis of, for example, a Monte Carlo method, a Kubelka-Munk theory, or a Beer-Lambert law.

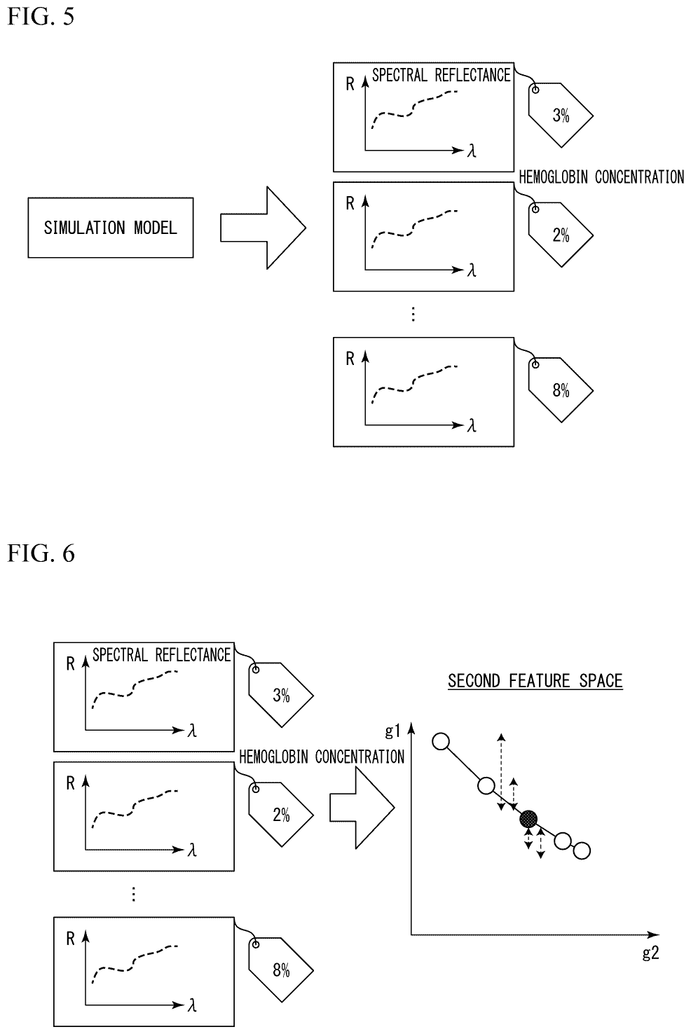

FIG. 5 is a diagram for describing a method for generating the pseudo spectral reflectance R\hat{ }. For example, the calculation function 122 inputs known pseudo internal component amount in a living organism (C_{Hb}, C_{H2O}, Cₘₑₗ, . . . ) to the decoder MDL2. In response to the input of the pseudo internal component amount in a living organism (C_{Hb}, C_{H2O}, Cₘₑₗ, . . . ), the decoder MDL2 simulates and outputs the pseudo spectral reflectance R\hat{ }. In other words, the calculation function 122 uses the decoder MDL2 to restore (decode) the pseudo spectral reflectance R\hat{ } based on the pseudo internal component amount in a living organism (C_{Hb}, C_{H2O}, Cₘₑₗ, . . . ). Restoration of the pseudo spectral reflectance R\hat{ } from the pseudo internal component amount in a living organism (C_{Hb}, C_{H2O}, Cₘₑₗ, . . . ) using the decoder MDL2 may be read as “reconstruction.”

In the shown example, the pseudo spectral reflectance R\hat{ } is calculated for each hemoglobin concentration C_{Hb}. For this reason, each pseudo spectral reflectance R\hat{ } is labeled with the hemoglobin concentration C_{Hb}, such as 3 (%), 2 (%), . . . , 8 (%).

Returning to the description of the flowchart, the generation function 123 then converts each of the plurality of pseudo spectral reflectances R\hat{ }, labeled with pseudo internal component amount in a living organism, into a pseudo feature vector using deep metric learning (step S108). In other words, the generation function 123 generates a pseudo feature vector based on each of the plurality of pseudo spectral reflectances RA using deep metric learning.

FIG. 6 is a diagram for describing a method of generating a pseudo feature vector. For example, the generation function 123 may use a pre-learned second deep metric learning model MDL3-2 to embed each of the plurality of pseudo spectral reflectances R\hat{ } into the second feature space as a pseudo feature vector. The second feature space may be a space different from the first feature space described above, and may have a different number of dimensions or a different base from the first feature space. In the shown example, the second feature space is represented as a two-dimensional space of g1 and g2, but is not limited to this. For example, the second feature space may be a high-dimensional space of three or more dimensions.

The second deep metric learning model MDL3-2 is a machine learning model that is learned to generate a pseudo feature vector based on a pseudo spectral reflectance R\hat{ } (to embed a pseudo spectral reflectance R\hat{ } into the second feature space) when the pseudo spectral reflectance R\hat{ } is input. The second deep metric learning model MDL3-2 is learned to dispose pseudo feature vectors with similar pseudo internal component amount in a living organism close to each other and dispose pseudo feature vectors with dissimilar pseudo internal component amount in a living organism far from each other in the second feature space when the pseudo spectral reflectance R\hat{ } is embedded into the second feature space. For example, the second deep metric learning model MDL3-2 is implemented by a neural network.

For example, on the basis of the pseudo internal component amount in a living organism, which is a label of the pseudo spectral reflectance R\hat{ }, identical pseudo internal component amounts in a living organism are determined to be a positive pair, and different pseudo internal component amounts in a living organism are determined to be a negative pair. The second deep metric learning model MDL3-2 is then learned under supervision according to the determined positive pair and negative pair. In other words, the second deep metric learning model MDL3-2 is learned to dispose pseudo feature vectors labeled with positive pairs closer to each other in the second feature space and to dispose pseudo feature vectors labeled with negative pairs far from each other.

The generation function 123 inputs the pseudo spectral reflectance R\hat{ } to the second deep metric learning model MDL3-2 learned in this manner. As a result, the second deep metric learning model MDL3-2 outputs a pseudo feature vector in response to the input of the pseudo spectral reflectance R\hat{ }, and further disposes similar pseudo feature vectors close to each other in the second feature space.

By adding another set of dataset of the pseudo spectral reflectance R\hat{ } and the pseudo internal component amount in a living organism through data expansion, the second deep metric learning model MDL3-2 may be learned by self-supervised learning with the two datasets as a positive pair, as in the first deep metric learning model MDL3-1.

As described above, the pseudo spectral reflectance R\hat{ } added through data expansion may be subjected to signal processing that reproduces the noise generated during capturing an image (such as uniformly reducing the pseudo spectral reflectance R\hat{ }).

Returning to the description of the flowchart, when a real spectral reflectance R is embedded as a real feature vector in the first feature space and a pseudo spectral reflectance R\hat{ } is embedded as a pseudo feature vector in the second feature space, the matching function 124 matches the real feature vector with the pseudo feature vector (step S110).

FIG. 7 is a diagram for describing feature vector matching. The matching function 124 moves the real feature vector embedded in the first feature space to the second feature space using, for example, optimal transport. For optimal transport, for example, Gromov-Wasserstein optimal transport may be used. Equation (1) represents an objective function of Gromov-Wasserstein optimal transport.

$$[ Equation 1 ]$$

$${min}_{P} {\sum }_{i , j , {i}^{′} , {j}^{′}} {\vert {D}_{i , {i}^{′}} - {D}_{j , {j}^{′}} \vert}^{2} {P}_{i , j} {P}_{{i}^{′} , {j}^{′}} \qquad ( 1 )$$

In the equation, D is a distance matrix (a difference between feature vectors), and P is a transport matrix. The matching function 124 transports the real feature vector embedded in the first feature space to the second feature space in accordance with Equation (1) so that a distance (D_{i,i′}-D_{j,j′}) between the real feature vectors is minimized before and after the real feature vector is transported from the first feature space to the second feature space. In other words, the matching function 124 transports the real feature vector embedded in the first feature space to the second feature space so that a distance D_{i,j′} between the real feature vectors in the first feature space is the same as a distance D_{j,j′} between the real feature vectors in the second feature space after the real feature vector is transported from the first feature space to the second feature space. Then, the matching function 124 matches the real feature vector with the pseudo feature vector in the second feature space.

Instead of Gromov-Wasserstein optimal transport, the matching function 124 may use other optimal transport such as unbalanced optimal transport (OT) or center of gravity optimal transport (barycenter OT) to match the feature vectors.

In addition, instead of optimal transport, the matching function 124 may use a gradient method such as a steepest descent method or a stochastic gradient descent method to match the feature vectors.

Returning to the description of the flowchart, the estimation function 125 next estimates the real internal component amount in a living organism for each of a plurality of pixels (real spectral reflectances R) included in the ROI on the edema image IMG on the basis of a matching result between the real feature vector and the pseudo feature vector (step S112).

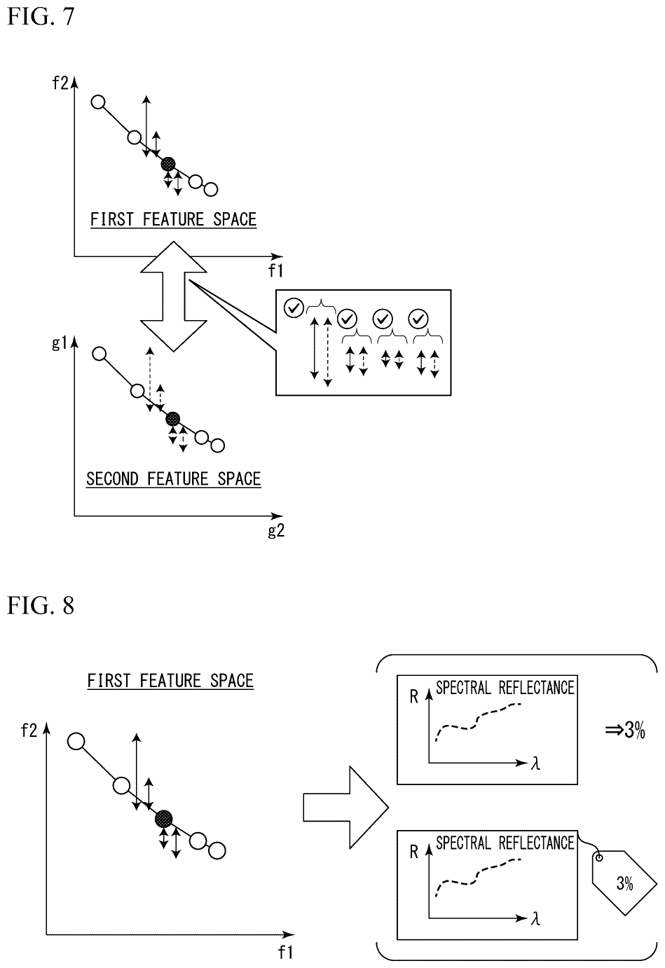

FIG. 8 is a diagram which shows an example of the matching result. For example, the estimation function 125 estimates the pseudo internal component amount in a living organism corresponding to the pseudo feature vector matched with the real feature vector as the real internal component amount in a living organism. As shown in FIG. 8, it is assumed that the hemoglobin concentration C_{Hb} labeled to the pseudo spectral reflectance R\hat{ } used to generate the pseudo feature vector is set to 3 [%]. In this case, the estimation function 125 estimates that the hemoglobin concentration C_{Hb} corresponding to the real spectral reflectance R used to generate the real feature vector matched with the pseudo feature vector is 3 [%]. In this manner, the real internal component amount in a living organism for each pixel included in the ROI are estimated.

Returning to the description of the flowchart, when the real internal component amount in a living organism of all pixels in the ROI is estimated, the output control function 126 next generates an image in which pixel values of all pixels have been replaced with the real internal component amount in a living organism (C_{Hb}, C_{H2O}, Cₘₑₗ, . . . ) (hereinafter referred to as a map of internal component amount in a living organism), and outputs this map of internal component amount in a living organism via the output interface 113 (step S114).

For example, the output control function 126 may display the map of internal component amount in a living organism on the display 113a. The output control function 126 may also transmit the map of internal component amount in a living organism to an external device (for example, a computer used by a doctor of a patient, or the like who is a subject of diagnosis) via the communication interface 111. This completes processing of this flowchart.

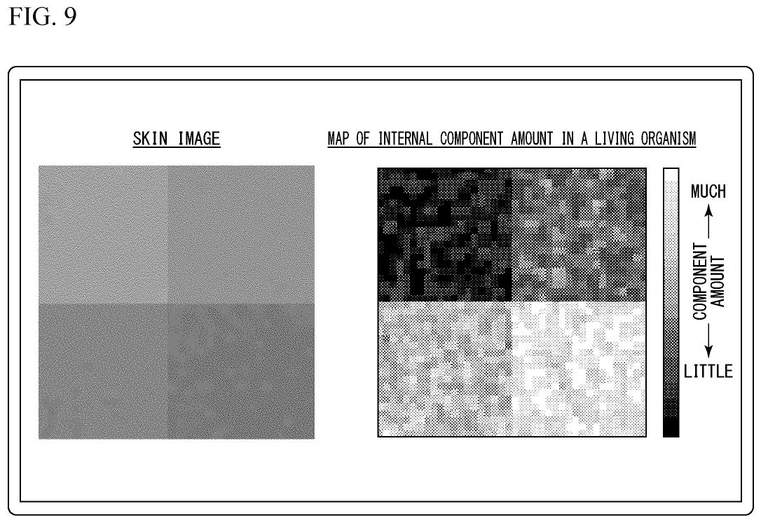

FIG. 9 is a diagram which shows an example of a screen of the display 113a on which the map of internal component amount in a living organism is displayed. For example, the screen of the display 113a may display the ROI of the edema image IMG (left in FIG. 9) and the map of internal component amount in a living organism of the ROI (right in FIG. 9) side by side. This allows a doctor or the like to diagnose a patient by comparing the real spectral reflectance R with the real internal component amount in a living organism.

According to the embodiment described above, the processing circuit 120 of the information processing apparatus 100 acquires the edema image IMG and acquires the real spectral reflectance R (an example of the “first observation data”) from each of the plurality of pixels included in the ROI of the edema image IMG.

The processing circuit 120 uses the first deep metric learning model MDL3-1 to generate a real feature vector (an example of the “first feature amount”) from the real spectral reflectance R.

The processing circuit 120 uses the second deep metric learning model MDL3-2 to generate a pseudo feature vector (an example of the “second feature amount”) based on the pseudo spectral reflectance R\hat{ } (an example of the “second observation data”) that has a nonlinear relationship with the pseudo internal component amount in a living organism (an example of the “second component amount”).

The processing circuit 120 matches the real feature vector with the pseudo feature vector. Then, the processing circuit 120 estimates a pseudo internal component amount in a living organism corresponding to the pseudo feature vector matched with the real feature vector as a real internal component amount in a living organism corresponding to the real feature vector (an example of the “first component amount”).

By embedding the real spectral reflectance R and the pseudo spectral reflectance R\hat{ } as feature vectors in the feature space using deep metric learning in this manner, and further matching the real feature vector and the pseudo feature vector in the feature space, it is possible to estimate information with high accuracy even when the relationship between the actual phenomenon and the simulation result cannot be mathematically expressed. For example, it is possible to estimate the extent of the internal component amount in a living organism, which indicates a degree of edema in the living organism, while reducing the influence of the discrepancy between the actual phenomenon and the simulation result.

In general, the real spectral reflectance R is likely to contain noise due to various factors such as reflected light from a skin surface, the reflection of shadows, and a variation in melanin concentration. When such noise is contained, the real spectral reflectance R may be large or small even if the hemoglobin concentration C_{Hb} in the skin is the same. In other words, even if the hemoglobin concentration C_{Hb} is the same, the sensitivity of the real spectral reflectance R will not match.

In contrast, in the present embodiment, the real spectral reflectance R is converted into a real feature vector by deep metric learning (the real spectral reflectance R is embedded in the first feature space), so that the effect of noise can be reduced. As a result, by matching feature vectors, it is possible to estimate with high accuracy the internal component amount in a living organism based on the edema image IMG, and furthermore, it is possible to more accurately diagnose a patient based on the real internal component amount in the living organism.

### Other Embodiments

Other embodiments will be described below. In the embodiment described above, the decoder MDL2 has been described as being implemented based on the Monte Carlo method, the Kubelka-Munk theory, or the Beer-Lambert law, but the present invention is not limited thereto. For example, the decoder MDL2 may be implemented by a neural network or a genetic algorithm, like the encoder MDL1.

Furthermore, in the embodiment described above, the decoder MDL2 has been described as estimating a real internal component amount in a living organism, such as C_{Hb}, C_{H2O}, and Cₘₑₗ, based on the real spectral reflectance R of each pixel of the edema image IMG, but the present invention is not limited thereto. For example, the processing circuit 120 may estimate a component amount including at least one of soft tissue, calcium, and iodine, based on the number of photons detected by a photon counting CT (Computed Tomography) device.

### Configuration of X-ray CT Apparatus

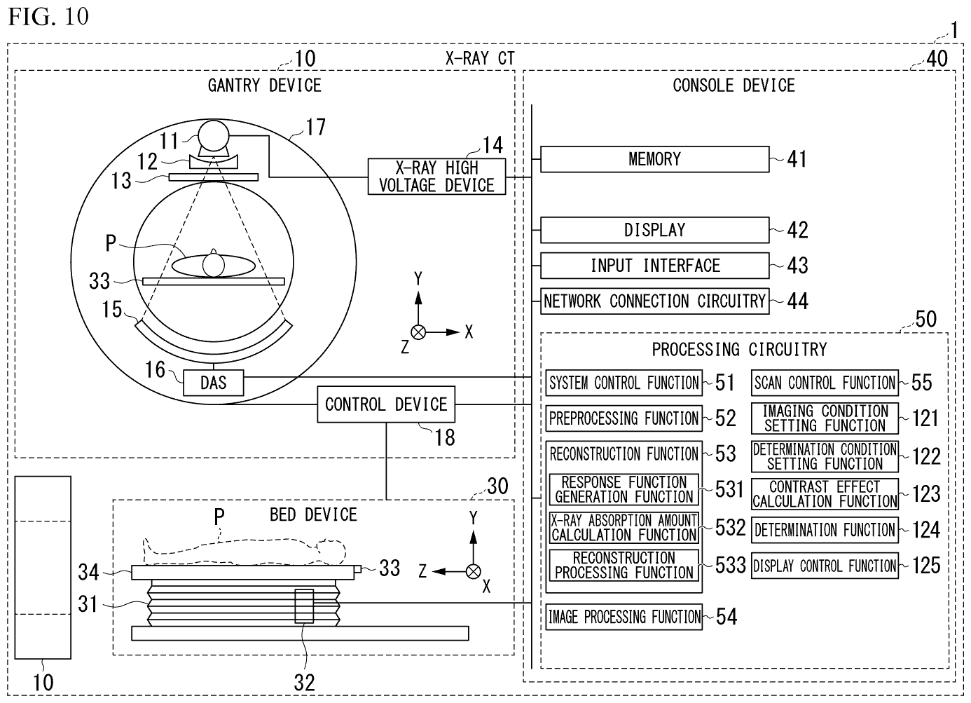

FIG. 10 is a diagram showing an example of an X-ray CT apparatus 1 according to an embodiment. The X-ray CT apparatus 1 of the embodiment is a photon counting computed tomography (CT) apparatus. A photon counting CT apparatus discriminates an inspection target substance through which X-rays have passed using a direct detector such as a semiconductor detector with excellent energy resolution.

The X-ray CT apparatus 1 includes, for example, a gantry device 10, a bed device 30, and a console device 40. Although FIG. 10 shows both a diagram of the gantry device 10 viewed in the Z-axis direction and a diagram of the gantry device 10 viewed in the X-axis direction for convenience of description, there is in fact one gantry device 10. In the present embodiment, the rotation axis of a rotating frame 17 in a non-tilt state or the longitudinal direction of a top plate 33 of the bed device 30 is defined as the Z-axis direction, an axis orthogonal to the Z-axis direction and parallel to the floor surface is defined as the X-axis direction, and a direction perpendicular to the Z-axis direction and perpendicular to the floor surface is defined as the Y-axis direction.

The gantry device 10 includes, for example, an X-ray tube 11, a wedge 12, a collimator 13, an X-ray high voltage device 14, an X-ray detector 15, a data acquisition system (hereinafter DAS) 16, the rotating frame 17, and a control device 18.

The X-ray tube 11 generates X-rays by radiating thermal electrons from a cathode (filament) to an anode (target) when a high voltage is applied thereto from the X-ray high voltage device 14. The X-ray tube 11 includes a vacuum tube. For example, the X-ray tube 11 is a rotating anode type X-ray tube that generates X-rays by radiating thermal electrons to a rotating anode.

The wedge 12 is a filter for adjusting a dose of X-rays radiated from the X-ray tube 11 to a subject P. The wedge 12 attenuates X-rays passing through the wedge 12 such that a distribution of the dose of X-rays radiated from the X-ray tube 11 to the subject P becomes a predetermined distribution. The wedge 12 is also called a wedge filter or a bow-tie filter. The wedge 12 is obtained by, for example, processing aluminum such that it has a predetermined target angle and a predetermined thickness.

The collimator 13 is a mechanism for narrowing down a radiation range of X-rays that have passed through the wedge 12. The collimator 13 narrows down the radiation range of X-rays by, for example, forming a slit by combining a plurality of lead plates. The collimator 13 may also be called an X-ray diaphragm. A narrowing range of the collimator 13 may be mechanically drivable.

The X-ray high voltage device 14 includes, for example, a high voltage generation device which is not shown and an X-ray control device which is not shown. The high voltage generation device has electric circuitry including a transformer, a rectifier, and the like and generates a high voltage to be applied to the X-ray tube 11. The X-ray control device controls the output voltage of the high voltage generation device in accordance with an amount of X-rays to be generated by the X-ray tube 11. The high voltage generation device may boost a voltage using the transformer described above or may boost the voltage using an inverter. The X-ray high voltage device 14 may be provided on the rotating frame 17 or may be provided on the side of a fixed frame (not shown) of the gantry device 10.

The X-ray detector 15 detects the intensity of X-rays generated by the X-ray tube 11 and incident through the subject P. The X-ray detector 15 outputs an electrical signal (which may be an optical signal or the like) corresponding to the detected intensity of X-rays to the DAS 16. The X-ray detector 15 has, for example, a plurality of X-ray detection element arrays. Each of the plurality of X-ray detection element arrays has a plurality of X-ray detection elements arranged in a channel direction along an arc having the focal point of the X-ray tube 11 as a center. The plurality of X-ray detection element arrays are arranged in a slice direction (column direction or row direction).

The X-ray detector 15 is, for example, a direct detection type detector. As the X-ray detector 15, for example, a semiconductor diode having electrodes attached to both ends of a semiconductor can be applied. X-ray photons incident on the semiconductor are converted into electron-hole pairs. The number of electron-hole pairs generated by incidence of one X-ray photon depends on the energy of the incident X-ray photon. Electrons and holes are each attracted to a pair of electrodes formed at both ends of the semiconductor. A pair of electrodes generates an electric pulse having a crest value corresponding to the charge of electron-hole pairs. A single electric pulse has a crest value corresponding to the energy of an incident X-ray photon.

The DAS 16 acquires count data indicating a count number of X-ray photons detected by the X-ray detector 15 for a plurality of energy bins, for example, according to a control signal from the control device 18. The count data for the plurality of energy bins corresponds to an energy spectrum with respect to X-rays incident on the X-ray detector 15 modified in accordance with the response characteristics of the X-ray detector 15. The DAS 16 outputs detection data based on digital signals to the console device 40. Detection data is a digital value of count data identified by a channel number and a row number of an X-ray detection element that is a generation source, and a view number indicating an acquired view. A view number is a number that changes according to rotation of the rotating frame 17 and is a number that is incremented according to rotation of the rotating frame 17, for example. Therefore, the view number is information indicating a rotation angle of the X-ray tube 11. A view period is a period that falls between a rotation angle corresponding to a certain view number and a rotation angle corresponding to the next view number.

The DAS 16 may detect switching of a view from a timing signal input from the control device 18, from an internal timer, or from a signal obtained from a sensor which is not shown. In a case where X-rays are continuously emitted from the X-ray tube 11 during full scanning, the DAS 16 acquires detection data groups for the entire circumference (for 360 degrees). In a case where X-rays are continuously emitted from the X-ray tube 11 during half scanning, the DAS 16 acquires detection data for a half circumference (for 180 degrees).

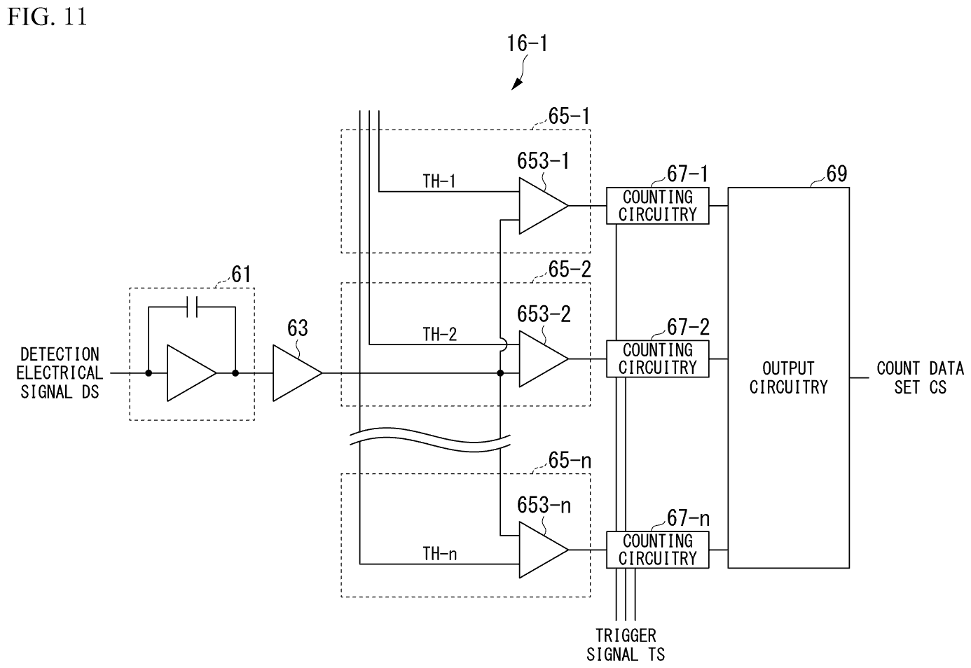

FIG. 11 is a diagram showing an example of a configuration of the DAS 16 according to the embodiment. The DAS 16 includes as many readout channels as the number of channels corresponding to the number of X-ray detection elements. These read channels are implemented in parallel in an integrated circuit, such as an application specific integrated circuit (ASIC). FIG. 11 shows only a configuration of a DAS 16-1 for one read channel.

The DAS 16-1 includes preamplifier circuitry 61, waveform shaping circuitry 63, a plurality of wave height discrimination circuitry 65, a plurality of counter circuitry 67, and output circuitry 69. The preamplifier circuitry 61 amplifies a detection electrical signal DS (current signal) from an X-ray detection element to which it is connected. For example, the preamplifier circuitry 61 converts the current signal from the X-ray detection element to which it is connected into a voltage signal having a voltage value (wave height value) proportional to the charge amount of the current signal. The waveform shaping circuitry 63 is connected to the preamplifier circuitry 61. The waveform shaping circuitry 63 shapes the waveform of the voltage signal from the preamplifier circuitry 61. For example, the waveform shaping circuitry 63 reduces the pulse width of the voltage signal from the preamplifier circuitry 61.

A plurality of counting channels corresponding to the number of energy bands (energy bins) are connected to the waveform shaping circuitry 63. In a case where n energy bins are set, n counting channels are provided in the waveform shaping circuitry 63. Each counting channel has wave height discrimination circuitry 65-n and counting circuitry 67-n.

Each of the wave height discrimination circuitry 65-n discriminates the energy of X-ray photons detected by the X-ray detection element, which is the wave height value of the voltage signal from the waveform shaping circuitry 63. For example, the wave height discrimination circuitry 65-n has comparison circuitry 653-n. A voltage signal from the waveform shaping circuitry 63 is input to one input terminal of each comparison circuitry 653-n. A reference signal TH (reference voltage value) corresponding to a different threshold value is supplied from the control device 18 to the other input terminal of each comparison circuitry 653-n.

For example, a reference signal TH-1 is supplied to comparison circuitry 653-1 for an energy bin bin1, a reference signal TH-2 is supplied to comparison circuitry 653-2 for an energy bin bin2, and a reference signal TH-n is supplied to comparison circuitry 653-n for an energy bin binn. Each reference signal TH has an upper limit reference value and a lower limit reference value. Each comparison circuitry 653-n outputs an electrical pulse signal in a case where the voltage signal from the waveform shaping circuitry 63 has a wave height value corresponding to the energy bin corresponding to each reference signal TH. For example, in a case where the wave height value of the voltage signal from the waveform shaping circuitry 63 corresponds to the energy bin bin1 (falls between the reference signals TH-1 and TH-2), the comparison circuitry 653-1 outputs an electrical pulse signal. On the other hand, in a case where the wave height value of the voltage signal from the waveform shaping circuitry 63 does not correspond to the energy bin bin1, the comparison circuitry 653-1 for the energy bin bin1 does not output an electrical pulse signal. Further, in a case where the wave height value of the voltage signal from the waveform shaping circuitry 63 corresponds to the energy bin bin2 (falls between the reference signals TH-2 and TH-3), for example, the comparison circuitry 653-2 outputs an electric pulse signal.

The counting circuitry 67-n counts electrical pulse signals from the wave height discrimination circuitry 65-n at a read cycle that matches a view switching cycle. For example, a trigger signal TS is supplied to the counting circuitry 67-n from the control device 18 at each view switching timing. By being triggered by supply of the trigger signal TS, the counting circuitry 67-n adds 1 to a count number stored in the internal memory each time an electrical pulse signal is input from the wave height discrimination circuitry 65-n. By being triggered by supply of the next trigger signal, the counting circuitry 67-n reads count number data (that is, the count data) stored in the internal memory and supplies the read count number data to the output circuitry 69. Further, the counting circuitry 67-n resets the count number accumulated in the internal memory to an initial value each time the trigger signal TS is supplied. In this manner, the counting circuitry 67-n counts the count number for each view.

The output circuitry 69 is connected to the counting circuitry 67-n for a plurality of readout channels mounted on the X-ray detector 15. The output circuitry 69 integrates count data from the counting circuitry 67-n for the plurality of readout channels for each of the plurality of energy bins to generate count data for the plurality of readout channels for each view. Count data of each energy bin is a set of count number data defined by a channel, a segment (column), and an energy bin. The count data of each energy bin is transmitted to the console device 40 on a view-by-view basis. The count data on a view-by-view basis is called a count data set CS.

The rotating frame 17 is an annular member that supports the X-ray tube 11, the wedge 12, the collimator 13, and the X-ray detector 15 while facing them. The rotating frame 17 is rotatably supported by a fixed frame around the subject P introduced therein. The rotating frame 17 also supports DAS 16. Detection data output by the DAS 16 is transmitted through optical communication from a transmitter having a light emitting diode (LED) provided on the rotating frame 17 to a receiver having a photodiode provided on a non-rotating part (e.g., fixed frame) of the gantry device 10 and forwarded to the console device 40 by the receiver. A method of transmitting the detection data from the rotating frame 17 to the non-rotating part is not limited to the above-described method using optical communication, and any non-contact transmission method may be employed. The rotating frame 17 is not limited to an annular member and may be a member such as an arm as long as it can support and rotate the X-ray tube 11 and the like.

The X-ray CT apparatus 1 is, for example, a rotate/rotate-type X-ray CT apparatus (third generation CT) in which both the X-ray tube 11 and the X-ray detector 15 are supported by the rotating frame 17 and rotate around the subject P, but not limited to thereto and may be a stationary/rotate-type X-ray CT apparatus (fourth generation CT) in which a plurality of X-ray detection elements arranged in an annular shape are fixed to a fixed frame and the X-ray tube 11 rotates around the subject P.

The control device 18 includes, for example, processing circuitry having a processor such as a central processing unit (CPU). The control device 18 receives an input signal from an input interface attached to the console device 40 or the gantry device 10 and controls operations of the gantry device 10, the bed device 30, and the DAS 16. For example, the control device 18 rotates the rotating frame 17 and tilts the gantry device 10. At the time of tilting the gantry device 10, the control device 18 rotates the rotating frame 17 about an axis parallel to the Z-axis direction on the basis of an inclination angle (tilt angle) input to the input interface. The control device 18 ascertains a rotation angle of the rotating frame 17 from the output of a sensor which is not shown, or the like. The control device 18 also controls energy bins (reference signal TH) of the DAS 16. The control device 18 may be provided in the gantry device 10 or may be provided in the console device 40.

The bed device 30 moves the subject P to be scanned placed thereon and enters the inside of the rotating frame 17 of the gantry device 10. The bed device 30 includes, for example, a base 31, a bed driving device 32, a top plate 33, and a support frame 34. The base 31 includes a housing that supports the support frame 34 such that the support frame 34 is movable in the vertical direction (Y-axis direction). The bed driving device 32 includes a motor and an actuator. The bed driving device 32 moves the top plate 33 along the support frame 34 in the longitudinal direction (Z-axis direction) of the top plate 33. The bed driving device 32 also moves the top plate 33 in the vertical direction (Y-axis direction). The top plate 33 is a plate-shaped member on which the subject Pis placed.

The bed driving device 32 may move not only the top plate 33 but also the support frame 34 in the longitudinal direction of the top plate 33. Contrary to the above, the gantry device 10 may be movable in the Z-axis direction, and the rotating frame 17 may be controlled to come around the subject P by moving the gantry device 10. Moreover, both the gantry device 10 and the top plate 33 may be configured to be movable. Further, the X-ray CT apparatus 1 may be an apparatus in which the subject P is scanned in a standing or sitting position. In this case, the X-ray CT apparatus 1 has a subject support mechanism in place of the bed device 30, and the gantry device 10 rotates the rotating frame 17 about the axial direction perpendicular to the floor surface.

The console device 40 includes, for example, a memory 41, a display 42, an input interface 43, network connection circuitry 44, and processing circuitry 50. In the embodiment, the console device 40 is described as being separate from the gantry device 10, but the gantry device 10 may include some or all of the components of the console device 40.

The memory 41 is realized by, for example, a semiconductor element such as a random access memory (RAM) or a flash memory, a hard disk, an optical disk, or the like. The memory 41 stores, for example, detection data, projection data, reconstructed image data, CT image data, information on the subject P, imaging conditions, and the like. The memory 41 stores count data regarding a plurality of energy bins transmitted from the gantry device 10, for example. Such data may be stored in an external memory with which the X-ray CT apparatus 1 can communicate instead of the memory 41 (or in addition to the memory 41). The external memory is controlled by a cloud server, for example, when the cloud server that manages the external memory receives a read/write request.

The display 42 displays various types of information. For example, the display 42 displays a medical image (CT image) generated by the processing circuitry, a graphical user interface (GUI) image for receiving various operations of an operator such as a doctor or an engineer, and the like. The display 42 is, for example, a liquid crystal display, a cathode ray tube (CRT), an organic electroluminescence (EL) display, or the like. The display 42 may be provided on the gantry device 10. The display 42 may be of a desktop type, or may be a display device (for example, a tablet terminal) capable of wireless communication with the main body of the console device 40.

The input interface 43 receives various input operations of the operator and outputs an electrical signal indicating the content of the received input operation to the processing circuitry 50. For example, the input interface 43 receives operations of inputting acquisition conditions at the time of acquiring detection data or projection data, reconstruction conditions at the time of reconstructing a CT image, image processing conditions at the time of generating post-processed images from CT images, energy bin setting conditions, and the like. For example, the input interface 43 is realized by a mouse, a keyboard, a touch panel, a drag ball, a switch, a button, a joystick, a camera, an infrared sensor, a microphone, and the like.

The input interface 43 may be provided in the gantry device 10. Further, the input interface 43 may be realized by a display device (for example, a tablet terminal) capable of wireless communication with the main body of the console device 40. In this specification, the input interface is not limited to those having physical operation parts such as a mouse and a keyboard. For example, examples of the input interface include electrical signal processing circuitry that receives an electrical signal corresponding to an input operation from external input equipment provided separately from the device and outputs the electrical signal to the control circuitry.

The network connection circuitry 44 includes, for example, a network card having a printed circuit board, a wireless communication module, or the like. The network connection circuitry 44 implements an information communication protocol in accordance with the form of a network to be connected.

The processing circuitry 50 controls the overall operation of the X-ray CT apparatus 1, the operation of the gantry device 10, and the operation of the bed device 30. The processing circuitry 50 includes, for example, a system control function 51, a preprocessing function 52, a reconstruction function 53, an image processing function 54, and a scan control function 55. The processing circuitry 50 further includes, for example, the acquisition function 121, the calculation function 122, the generation function 123, the matching function 124, the estimation function 125, and the output control function 126 described above.

These components are realized by, for example, a hardware processor (computer) executing a program (software) stored in the memory 41. The hardware processor means, for example, circuitry such as a CPU, a graphics processing unit (GPU), an application specific integrated circuit (ASIC), or a programmable logic device (e.g., a simple programmable logic device (SPLD) or a complex programmable logic device (CPLD), or a field programmable gate array (FPGA)).

Instead of storing the program in the memory 41, the program may be directly embedded in the circuit of the hardware processor. In this case, the hardware processor realizes the functions by reading and executing the program embedded in the circuit. The hardware processor is not limited to being configured as a single circuit and may be configured as one hardware processor by combining a plurality of independent circuits to realize each function. Further, a plurality of components may be integrated into one hardware processor to realize each function.

Each component of the console device 40 or the processing circuitry 50 may be distributed and realized by a plurality of pieces of hardware. The processing circuitry 50 may be realized by a processing device capable of communicating with the console device 40 instead of being a component included in the console device 40. The processing device is, for example, a workstation connected to one X-ray CT apparatus or a device (e.g., cloud server) that is connected to a plurality of X-ray CT apparatuses and collectively executes processing equivalent to that of the processing circuitry 50 which will be described below.

The system control function 51 controls various functions of the processing circuitry 50 on the basis of input operations received through the input interface 43. The system control function 51 sets energy bins, for example. The system control function 51 outputs set energy bin setting conditions to the control device 18.

The preprocessing function 52 performs preprocessing such as offset correction processing, inter-channel sensitivity correction processing, and beam hardening correction on detection data output from the DAS 16.

The reconstruction function 53 reconstructs a photon counting CT image of the subject P on the basis of detection data (count data). The reconstruction function 53 includes, for example, a response function generation function 531, an X-ray absorption amount calculation function 532, and a reconstruction processing function 533. The response function generation function 531 generates response function data representing detector response characteristics. For example, the response function generation function 531 measures a response (i.e., detected energy and detected intensity) of a standard detection system to a plurality of monochromatic X-rays having a plurality of incident X-ray energies through predictive calculations, experiments, and combinations of predictive calculations and experiments, and generates a response function on the basis of measured values of the detected energy and the detected intensity. Further, the response function generation function 531 may generate response function data on the basis of actual measurement values acquired in calibration or the like. The response function defines a relationship between a detected energy for each incident x-ray and an output response of the system. For example, the response function defines a relationship between a detected energy and a detected intensity for each incident x-ray. The generated response function data is stored in the memory 41.

The X-ray absorption amount calculation function 532 calculates an X-ray absorption amount with respect to each of a plurality of ground substances on the basis of count data regarding a plurality of energy bins, the energy spectrum of X-rays incident on the subject P, and the response function stored in the memory 41. The X-ray absorption amount calculation function 532 can calculate an X-ray absorption amount having no influence on the response characteristics of the X-ray detector 15 and the DAS 16 by calculating the X-ray absorption amount using the response function on the basis of the count data and the energy spectrum of the X-rays incident on the subject P. Processing of obtaining the X-ray absorption amount for each ground substance in this manner is also called substance discrimination. Any substance such as calcium, calcification, bone, fat, muscle, air, organ, lesion, hard tissue, soft tissue, and contrast agent can be set as a ground substance. The type of ground substance to be calculated may be determined in advance by an operator or the like via the input interface 43. An X-ray absorption amount indicates the amount of X-rays absorbed by a ground substance. For example, an X-ray absorption amount is defined by a combination of an X-ray attenuation coefficient and an X-ray transmission path length.

The reconstruction processing function 533 reconstructs a photon counting CT image representing a spatial distribution of a ground substance to be imaged among a plurality of ground substances on the basis of the X-ray absorption amount for each of the plurality of ground substances calculated by the X-ray absorption amount calculation function 532 and stores the generated CT image data in the memory 41. The ground substance to be imaged may be of one type or of a plurality of types. The type of ground substance to be imaged may be determined by an operator or the like via the input interface 43.

The reconstruction function 53 generates data from which a plurality of types of contrast-enhancing substances can be discriminated on the basis of detection results obtained by monitoring scanning performed on a subject into which the plurality of types of contrast-enhancing substances have been injected.

The image processing function 54 converts a CT image data into three-dimensional image data or cross-sectional image data of an arbitrary cross section through a known method on the basis of an input operation received through the input interface 43. Conversion to three-dimensional image data may be performed by the preprocessing function 52.

The scan control function 55 controls detection data acquisition processing in the gantry device 10 by instructing the X-ray high voltage device 14, the DAS 16, the control device 18, and the bed driving device 32. The scan control function 55 performs control for monitoring scanning and main scanning which will be described later. Further, the scan control function 55 controls the operation of each unit at the time of imaging for acquiring positioning images and capturing an image used for diagnosis.

With this configuration, the processing circuitry 50 of the X-ray CT apparatus 1 (photon counting CT apparatus) performs a CT scan on the subject P by causing the X-ray tube 11 irradiate X-rays.

The processing circuit 50 acquires the number of photons (another example of the “first observation data”) detected by the X-ray detector 15 throughout the CT scan.

The processing circuit 50 generates a real feature vector based on the actually measured number of photons using the first deep metric learning model MDL3-1.

The processing circuit 50 generates a pseudo feature vector based on the pseudo number of photons (another example of the “second observation data”) that has a nonlinear relationship with known component amounts such as soft tissue, calcium, and iodine (another example of “second component amount”) using the second deep metric learning model MDL3-2.

The processing circuit 50 matches the pseudo feature vector with the real feature vector. The processing circuit 50 then estimates the known component amount corresponding to the pseudo feature vector matched with the real feature vector as a component amount corresponding to the real feature vector (another example of the “first component amount”). Through such processing, it is possible to estimate component amounts such as soft tissue, calcium, iodine, and the like with high accuracy based on the actually measured number of photons.

In addition, the processing circuit 120 of the information processing apparatus 100 in the embodiment described above, may estimate an elasticity of a biological tissue irradiated with ultrasonic waves from echo signals measured by an ultrasound diagnostic device.

More specifically, the processing circuit 120 acquires the echo signals measured by the ultrasound diagnostic device (another example of the “first observation data”).

The processing circuit 120 uses the first deep metric learning model MDL3-1 to generate a real feature vector based on the actually measured echo signals.

The processing circuit 120 uses the second deep metric learning model MDL3-2 to generate a pseudo feature vector based on a pseudo echo signal (another example of the “second observation data”) that has a nonlinear relationship with the known elasticity of a biological tissue (another example of the “second component amount”).

The processing circuit 120 matches the real feature vector with the pseudo feature vector. Then, the processing circuit 120 estimates the elasticity corresponding to the pseudo feature vector matched with the real feature vector as the elasticity corresponding to the real feature vector (another example of the “first component amount”). Through such processing, it is possible to estimate the elasticity of a biological tissue with high accuracy based on the actually measured echo signals.

The processing circuit 120 may also estimate electrical characteristic parameters (such as electrical conductivity and dielectric constant) of biological tissue from the magnetic field observed by the magnetic resonance imaging device.

More specifically, the processing circuit 120 acquires a signal or data (for example, a magnetic resonance signal, magnetic resonance data, or k-space data) indicating the magnetic field observed by the magnetic resonance imaging device. The magnetic resonance signal, magnetic resonance data, and k-space data are other examples of the “first observation data.”

The processing circuit 120 generates a real feature vector based on the actually measured magnetic resonance signal, and the like, using the first deep metric learning model MDL3-1.

The processing circuit 120 uses the second deep metric learning model MDL3-2 to generate a pseudo feature vector based on pseudo magnetic resonance signals (another example of the “second observation data”) that have a nonlinear relationship with known electrical characteristic parameters (another example of the “second component amount”).

The processing circuit 120 matches the pseudo feature vector with the real feature vector. The processing circuit 120 then estimates electrical characteristic parameters corresponding to the pseudo feature vector matched with the real feature vector as electrical characteristic parameters corresponding to the real feature vector (another example of the “first component amount”). Through such processing, it is possible to estimate electrical characteristic parameters (such as electrical conductivity and dielectric constant) of a biological tissue with high accuracy based on actually measured magnetic resonance signals, magnetic resonance data, k-space data, or the like.

In addition, the processing circuit 120 may estimate component amounts of an object of non-destructive testing based on echo signals of electromagnetic waves or ultrasound observed by non-destructive testing using electromagnetic waves, ultrasonic waves, or the like. For example, the object of non-destructive testing may be concrete, and the component amounts may be hardness, a strain, a carbon content, a vibration propagation speed, and the like.

More specifically, the processing circuit 120 acquires echo signals (another example of the “first observation data”) of electromagnetic waves or ultrasonic waves observed by non-destructive testing.

The processing circuit 120 generates a real feature vector based on the actually measured echo signals using the first deep metric learning model MDL3-1.

The processing circuit 120 generates a pseudo feature vector based on a pseudo echo signal (another example of the “second observation data”) that has a nonlinear relationship with known component amounts such as hardness, a strain, and a carbon content (another example of the “second component amount”) using the second deep metric learning model MDL3-2.

The processing circuit 120 matches the pseudo feature vector with the real feature vector. Then, the processing circuit 120 estimates a component amount corresponding to the pseudo feature vector matched with the real feature vector as a component amount corresponding to the real feature vector (another example of the “first component amount”). Through such processing, it is possible to estimate component amounts of concrete, and the like with high accuracy based on the echo signals of electromagnetic waves or ultrasonic waves observed by non-destructive testing.

Although several embodiments have been described, these embodiments are presented as examples and are not intended to limit the scope of the invention. These embodiments can be implemented in various other forms, and various omissions, substitutions, and modifications can be made within a range not departing from the gist of the invention. These embodiments and their modifications are included within the scope of the invention and its equivalents as set forth in the claims, as well as within the scope and gist of the invention.

What is claimed is:

1. An X-ray computed tomography apparatus comprising processing circuitry configured to:
acquire a number of photons by performing a CT scan on a target event;
generate a first feature amount from the number of photons;
match the first feature amount to second feature amounts generated using a machine learning model from the number of photons of a reference event, the number of photons of the reference event being a number of photons generated from known component amounts of the reference event and having a nonlinear relationship with the known component amounts; and
estimate the known component amounts corresponding to the second feature amount matched to the first feature amount as component amounts of the target event.

2. An information processing method comprising:
acquiring first observation data, which is observation data of a target event;
generating a first feature amount from the first observation data using deep metric learning;
matching the first feature amount to second feature amounts generated using deep metric learning from the second observation data, which is observation data of a reference event generated from second component amounts, which are known component amounts of the reference event, and has a nonlinear relationship with the second component amounts; and
estimating the second component amounts corresponding to the second feature amount matched to the first feature amount as first component amounts, which are component amounts of the target event.

3. The information processing method according to claim 2,
wherein the first feature amount is matched to the second feature amount using optimal transport.

4. The information processing method according to claim 3,
wherein the optimal transport transports the first feature amount so that a distance between the first feature amount is the same before and after transporting the first feature amount from a first feature space in which the first feature amount is distributed to a second feature space in which the second feature amount is distributed, and
the first feature amount is matched with the second feature amount in the second feature space.

5. The information processing method according to claim 2,
wherein the first feature amount is matched to the second feature amount using a gradient method.

6. The information processing method according to claim 2,
wherein the first observation data and the second observation data include a spectral reflectance, and
the first component amounts and the second component amounts include any one of a hemoglobin concentration, a water concentration, and a melanin concentration.

7. The information processing method according to claim 2,
wherein the first observation data and the second observation data include the number of photons detected by a photon counting CT device, and
the first component amounts and the second component amounts include any one of soft tissue, calcium, and iodine.

8. The information processing method according to claim 2,
wherein the first observation data and the second observation data include an echo signal measured by an ultrasound diagnostic device, and
the first component amounts and the second component amounts include an elasticity of biological tissue.

9. The information processing method according to claim 2,
wherein the first observation data and the second observation data include a magnetic field observed by a magnetic resonance imaging device, and
the first component amounts and the second component amounts include electrical characteristic parameters of biological tissue.

10. An information processing apparatus comprising:
a processing circuit configured to acquire first observation data, which is observation data of a target event,
generate a first feature amount from the first observation data using deep metric learning.
match the first feature amount to second feature amounts generated using deep metric learning from the second observation data, which is observation data of a reference event generated from second component amounts, which are known component amounts of the reference event, and has a nonlinear relationship with the second component amounts, and
estimate the second component amounts corresponding to the second feature amount matched to the first feature amount as first component amounts, which are component amounts of the target event.

11. A computer-readable non-transitory storage medium which has stored a program causing a computer to execute:
acquiring first observation data, which is observation data of a target event, generating a first feature amount from the first observation data using deep metric learning,
matching the first feature amount to second feature amounts generated using deep metric learning from the second observation data, which is observation data of a reference event generated from second component amounts, which are known component amounts of the reference event, and has a nonlinear relationship with the second component amounts, and
estimating the second component amounts corresponding to the second feature amount matched to the first feature amount as first component amounts, which are component amounts of the target event.
